(19) United States
(12) Patent Application Publication
(10) Pub. No.: US 2009/0105545 A1
(43) Pub. Date: Apr. 23, 2009

(54) Apparatus and method for facilitating the implantation of a medical device

(75) Inventors: Abram D. Janis, Valencia, CA (US); Claude A. Vidal, Santa Barbara, CA (US); Russell J. Redmond, Goleta, CA (US)

Correspondence Address: ARTHUR FREILICH;FREILICH, HORNBAKER & ROSEN, 20555 DEVONSHIRE ST. #372, CHATSWORTH, CA, 91311, US

(73) Assignee: Medical Research Products-B, Inc.

(21) Appl. No.: 12/287,130

(22) Filed: Oct. 6, 2008

Related U.S. Application Data
(60) Provisional application No. 60/999,480, filed on Oct. 17, 2007.

(51) Int. Cl. A61B 17/32; A61B 19/00

(52) U.S. Cl. 600/184; 128/898; 606/167

(57) ABSTRACT

An apparatus and method for facilitating the implantation of a medical device through an incision so as to promote soft tissue ingrowth into a biocompatible porous layer, e. g, titanium, carried on the periphery of the medical device. The method utilizes an incision (either percutaneous or subcutaneous) which is intentionally undersized by 10-20% relative to the width dimension of the porous layer. Accordingly, a physician must stretch the surrounding tissue to maximize the size of the opening to insert the device. Because the opening is undersized relative to the porous layer, the surrounding tissue remains physically stressed, i.e., radially and/or circumferentially, and acts to enhance cell proliferation and healing. A surgical cutting tool is preferably provided to assist the physician to form a properly dimensioned opening.

### RELATED APPLICATION

This application claims priority based on U.S. Provisional Application 60/999,480 filed on 17 Oct. 2007.

### FIELD OF THE INVENTION

This invention relates generally to medical technology and more particularly to a method and apparatus for facilitating the implantation of medical devices.

### BACKGROUND OF THE INVENTION

A variety of medical procedures involve implanting a device through a percutaneous or subcutaneous incision into a patient's soft tissue. If the device is intended to remain in situ over a long period of time, it is desirable that the tissue surrounding the incision grow toward, and seal against, the device. To encourage such sealing and the forming of an infection resistant barrier, it has been proposed that the device periphery carry a layer, or band, of porous material, e.g., a biocompatible mesh, to promote tissue ingrowth.

### SUMMARY OF THE INVENTION

The present invention is directed to an apparatus and method for facilitating the implantation of a medical device through a percutaneous or subcutaneous incision. More particularly, the invention is directed to an apparatus and method which promotes soft tissue ingrowth into porous biocompatible material, e.g., titanium, carried on the periphery of a medical device.

The present invention is based on the recognition that the rate and/or extent of soft tissue ingrowth can be enhanced by increasing the mechanical interaction between a device porous layer and a patient's soft tissue. This is accomplished in accordance with the invention by forming an incision (either percutaneously or subcutaneously) which is intentionally undersized relative to the width dimension of the porous layer. As a consequence, in order to insert the device into the opening formed by the incision, the physician should first manually stretch the surrounding tissue to maximize the size of the opening. After the device is placed in the opening and the manual stretching terminated, the surrounding tissue relaxes around the porous layer. However, because the opening is undersized relative to the porous layer, the surrounding tissue is physically stressed, i.e., radially and/or circumferentially, which acts to enhance cell proliferation and healing.

In accordance with the invention, the incision should be undersized by 10-20%, e.g., if the device porous layer outer diameter (OD) is W then an opening should be formed which has a width between 0.8W and 0.9W. In a preferred embodiment, the incision is formed to provide an opening, having a width, which is about 15% smaller than the width W defined by the device porous layer. For example, if the device carries a porous layer having a width, i.e., outer diameter (OD), of 0.310″, it is desirable to provide a relaxed incision opening of about 0.260″.

A surgical cutting tool is preferably provided in accordance with the invention to assist the physician to form a properly dimensioned opening. A preferred cutting tool includes a handle carrying a precisely dimensioned cutting edge, e.g., a forwardly projecting blade. A preferred blade defines a cutting edge includes first and second edge portions which diverge rearwardly from a pointed blade front end. The rear edges of the first and second edge portions are spaced to define a maximum width of 0.9W.

### BRIEF DESCRIPTION OF THE FIGURES

FIG. 1 is a schematic representation generally depicting a catheter assembly used in accordance with the invention for percutaneously implanting a catheter for an exemplary hemodialysis application;

FIG. 2 is an isometric view of a preferred catheter assembly;

FIG. 3 is an exploded view of the assembly of FIG. 2 showing a catheter in phantom together with a protective sheath, an anchor, a sleeve carrying a layer of porous material, an annular seal, and a locking member;

FIG. 4A is a sectional view taken substantially along the plane of 4A-4A of FIG. 2;

FIG. 4B is a sectional view taken substantially along the plane 4B-4B of FIG. 4A showing the locking member in its unlocked state;

FIG. 4C is a sectional view similar to FIG. 4B showing the locking member in its locked state clamped by suture or wire;

FIGS. 4D and 4E show exemplary spring clips which can be alternatively used for clamping the locking member in its locked state;

FIG. 5 is a plan view of the protective sheath of FIG. 3;

FIG. 6 is a sectional view taken substantially along the plane 6-6 of FIG. 5 particularly showing a perforated score line;

FIG. 7 is a drawing showing a dimensioned incision formed in accordance with the present invention;

FIG. 8 is a sectional view showing the assembly of FIG. 2 accommodated in the stretched opening of FIG. 7;

FIG. 9 is an isometric view of an exemplary surgical cutting tool for forming the incision of FIG. 7;

FIG. 10 is a side view of the cutting tool of FIG. 9;

FIG. 11 is an end view of the cutting tool of FIG. 10; and

FIG. 12 is a sectional view taken substantially along the plane 12-12 of FIG. 10.

### DETAILED DESCRIPTION

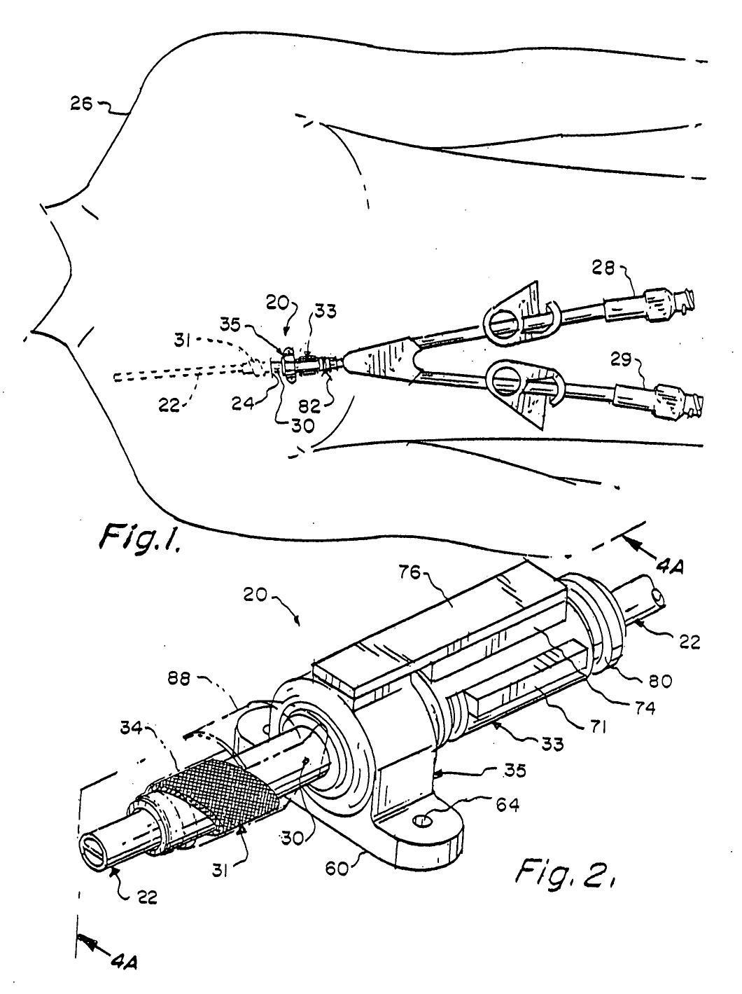

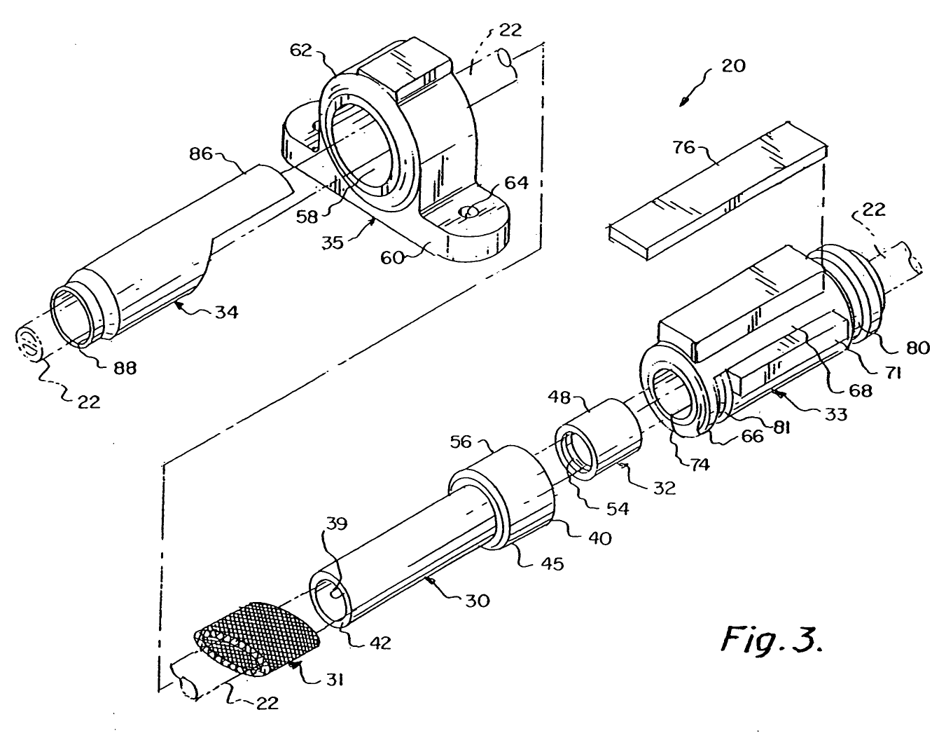

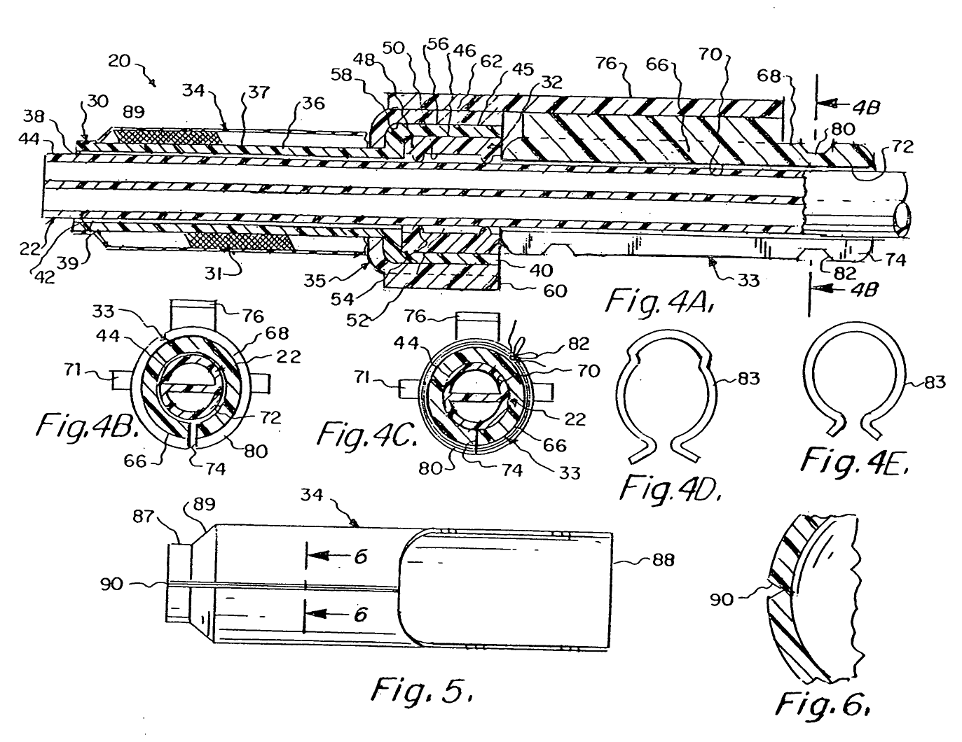

Various medical regimens relating, for example, to hemodialysis drug infusion, plasmapheresis, etc., use a percutaneously implanted conduit for conveying fluid and/or electric signals to/from an interior body site. FIGS. 1-6 of this application essentially duplicate corresponding figures of published Application US-2007-0149949-A (which application is incorporated herein by reference) which illustrate an exemplary assembly 20 for percutaneously implanting a catheter 22 through an incision 24 in a patient 26 undergoing an exemplary hemodialysis procedure. In such a procedure, a dual lumen catheter 22 is typically used with the two lumens being respectively coupled to separate exterior flow couplers 28 and 29. FIGS. 2-4 show the primary elements of the assembly 20 including a sleeve 30 carrying a porous layer 31, a sealing device 32, and a locking member 33. The assembly 20 preferably also may include an optional protective sheath 34 and an anchor 35 for anchoring the assembly 20 to a patient's outer skin surface. The sleeve 30 preferably comprises a substantially rigid tubular member formed of biocompatible material, e.g., titanium. The sleeve 30 includes a peripheral wall 36 (FIG. 4) having an outer surface 37 and an inner surface 38. The inner surface 38 surrounds an interior passageway 39 extending axially from a sleeve first, or proximal, end 40 to a sleeve second, or distal, end 42.

The sleeve 30 is shown mounted on a catheter 22 extending axially through the passageway 39. The catheter outer surface 44 and passageway wall surface 38 are closely dimensioned but with sufficient clearance therebetween to enable the catheter to slide axially and rotate in the passageway 39. The sleeve 30 proximal end 40 is preferably enlarged at 45 to form an interior recess 46 for accommodating the sealing device 32. The sealing device 32 preferably comprises an annular member 48 formed of a soft flexible material, e.g., silicone. The seal member 48 defines an inner peripheral surface 50 surrounding an interior bore 52 which is contiguous with sleeve passageway 39. At least one flexible annular nib 54 extends radially into the bore 52 for contacting and sealing against the catheter outer surface 44.

The enlarged sleeve end 45 has an outer peripheral surface 56 dimensioned to closely fit into bore 58 of anchor 35. The anchor 35 comprises a base portion 60 supporting a ferrule portion 62 which defines the bore 58. The anchor base portion 62 is provided with holes 64 to facilitate the suturing of anchor 35 to the patient's skin.

The locking member 33 preferably comprises a split ring formed of soft flexible material, e.g., silicone. More particularly, the locking member 33 is comprised of a peripheral wall 66 having an outer surface 68 and an inner surface 70 surrounding an interior bore 72. The wall 66 is longitudinally split at 74. The wall outer surface 68 is preferably provided with one or more strap pads 71 for securing the locking member 33 to the anchor 35 and/or sleeve 30 using one or more straps 76. The locking member outer wall surface is provided with a proximal annular groove 80 for accommodating suture thread or an appropriately shaped spring clip which can be used by a physician to compress the locking member 33 around the catheter 22. Preferably, a distal annular groove 81 is also provided.

The locking member 33 is configured so that in its natural unlocked state (FIG. 4B), the interior bore 72 is sufficiently large to permit the catheter 22 to slide axially and rotate in the bore 72 and through the sleeve passageway 39. The physician can compress the locking member wall 66 around the catheter to frictionally engage the locking member inner surface 70 against the catheter outer surface 44 to thus lock the catheter outer surface 44 to the sleeve 30 to prevent any relative movement therebetween. This locked state can be maintained by tying suture thread 82 around the locking member wall in grooves 80, 81. Of course, the thread 82 can be readily cut when it is desired to release the locked state to allow the catheter to be repositioned and/or replaced. FIGS. 4D and 4E depict exemplary spring clips 83 which can be alternatively placed in the grooves 80, 81 in lieu of thread 82 for clamping the locking member in its locked state.

The layer of porous material 31, e.g., titanium mesh, having a pore size within a range of 50 to 200 microns with a porosity of 60 to 95% (as described in U.S. application Ser. No. 10/821,383), is mounted around the outer surface 37 of sleeve 30, close to the sleeve distal end 42. In use, it is intended that the sleeve distal end be inserted through an incision 24 (FIG. 1) in the patient's skin to position the porous layer 31 just below the patient's epidermal skin layer and in contact with the patient's dermal layer. Note in FIG. 2 that the porous layer 31 is preferably oriented diagonally with respect to the axis of sleeve 30 to better conform to the patient's skin contour. This orientation optimizes contact between the porous layer 31 and the patient's subcutaneous tissue to promote, over time, soft tissue ingrowth into the porous layer. This tissue ingrowth acts to firmly anchor the sleeve in place and to form an infection resistant barrier around sleeve 30. This barrier may be enhanced by incorporating antimicrobial and/or ant-inflammatory constituents into the porous layer 31. For example, silver containing compounds and/or antibiotic eluting coatings can be used as antimicrobial agents and steroids can be used as ant-inflammatory agents.

The aforementioned protective sheath 34 is preferably formed of thin flexible tubular material (e.g., 0.010″ wall FEP tubing) and is intended to be mounted around sleeve 30 and porous layer 31 prior to use to avoid injuring the patient's tissue when the sleeve distal end 42 is inserted through the incision 24. As described in U.S. application Ser. No. 11/708,445, the sheath 34 is removed from the sleeve 30 by the physician as the sleeve and porous layer are being inserted through the incision.

More particularly, the sheath 34 is preferably configured as a substantially tubular, e.g., cylindrical, body 86 having a distal collar 87 and a proximal elongate pull tab 88. An outwardly tapering section 89 extends from the collar 87 to the main body portion 86. Note that the collar 87 and distal portion of section 89 have a diameter smaller than that of the porous layer 31. For example only, the sleeve 30 may have an outer diameter of 0.250 inches, the porous layer 31 an outer diameter of 0.310 inches and the collar 87 an inner diameter of 0.193 inches. An axially oriented score, or perforated line 90 is preformed through the collar 87, the tapering section 89 and the body portion 86 to facilitate the physician peeling the sheath 34 from the sleeve 30. Note in FIG. 4A that the sheath fits tightly around the periphery of sleeve 30 and porous layer 31 and that the tapering section 89 is positioned distally of the porous layer 31. In use, the physician is able to readily peel the sheath from the sleeve with one hand by rolling, or winding, the elongate tab to pull the sheath axially in a proximal direction. Peeling occurs because as the sheath is pulled proximally, the tapering section 89 and collar 87 have to move past the larger diameter porous layer 31 which action causes the sheath to tear along score line 90 allowing it to be easily stripped from the sleeve 30.

In the preferred catheter assembly illustrated in FIG. 24A, the sleeve 30 comprises a rigid titanium tube characterized substantially as follows:

| | |
|---|---|
| overall length | 1.135 inches |
| proximal end 45 length | .250 inches |
| passageway 39 ID | .200 inches |
| end 45 ID | .313 inches |
| sleeve 30 wall thickness | .025 inches |
| porous material 31 OD | .310 inches |
| nib 54 ID | .170 inches |

Attention is now directed to FIG. 7 which illustrates the aforementioned incision 24 dimensioned in accordance with the present invention. That is, in accordance with the present invention, the incision 24 is formed to provide an opening 100 having a width which is 10% to 20% less than the width W (i.e., outer dimeter, OD) of the porous layer 31. In order to insert the sleeve with porous layer 31 into the opening 100, the physician typically manually stretches the tissue surrounding the opening. The insertion is facilitated by the presence of sheath 34 which is removed by the physician in the course of device insertion. After the device, i.e., sleeve 30 and porous layer 31, enters the opening 100, the physician can terminate the manual stretching to allow the surrounding tissue to relax toward the porous layer 31. Even in this relaxed state, however, because the opening 100 is undersized relative to the width of the porous layer 31, the surrounding tissue remains stressed, e.g., radially and/or circumferentially, which acts to enhance cell proliferation and ingrowth into the porous layer.

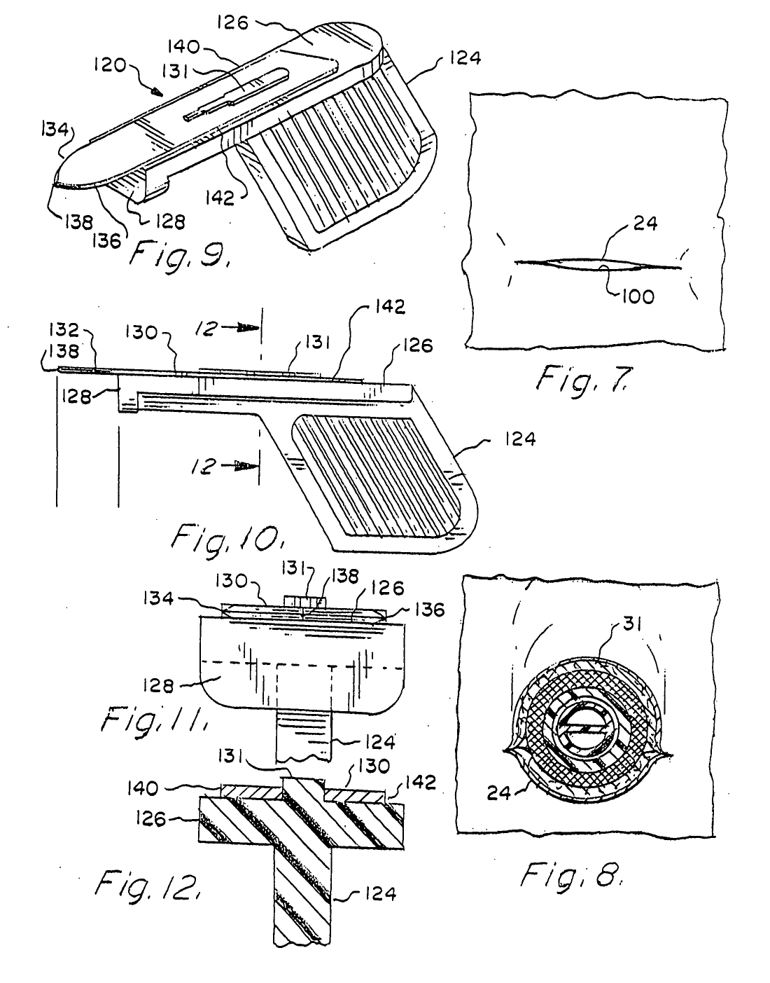

In order to assist the physician to form a closely dimensioned undersized opening in accordance with the invention, it is preferable to provide a surgical cutting tool having a cutting edge dimensioned to the desired opening size. FIGS. 9-12 illustrate one such preferred surgical cutting tool 120 for forming a closely dimensioned incision 24. The tool 120 is comprised of a handle 124 having a flat upper surface 126 defining a forward face 128. A blade 130 is mounted on the surface 126 using, for example, a surface protuberance 131 extending into a keyway in the blade. The blade 130 has a front end 132 projecting beyond the handle face 128 comprised of first and second substantially straight cutting edge portions 134, 136. The portions 134, 136 diverge rearwardly from a pointed end 138. The cutting edge portions 134, 136 extend rearwardly and blend into blade parallel sides 140, 142. The maximum width of the cutting edge portions, i.e., the spacing between sides 140 and 142, is selected to be between 80% and 90% of the width W of the device to be inserted.

In use, a physician will pierce the patient's skin with the blade point 138, pushing the blade straight inwardly until stopped by face 128. That is, the spacing between pointed end 138 and face 128 will define the depth (e.g., 0.39″) of the incision 24 formed by the blade 130. By pushing straight inwardly, the physician is able to create a clean closely dimensioned incision 24 to form opening 100. By selecting the blade width to be between 80 and 90% of the width of the device to be inserted, the physician will form a closely undersized opening. The physician will then manually stretch the skin around opening in order to insert the device. After insertion and after termination of the manual stretching, the surrounding tissue will elastically retract against the device but will remain physically stressed by the relatively oversized device. The residual stress, or tension, in the surrounding tissue acts to stimulate healing and promote tissue ingrowth into the device porous layer 31. In addition to the effects of circumferential strain on cell proliferation or hypoxic signaling upregulating angiogenic response, a tighter incision in accordance with the invention may decrease the volume of the underlying subcutaneous pocket thus stabilizing the implanted device and reducing foreign body response to movement of the device.

Although the method disclosed herein has been described primarily with regard to a percutaneously implanted sleeve carrying a porous layer, it is emphasized that the invention also finds utility with regard to a variety of different medical procedures for implanting devices into soft tissue. Further, although an exemplary preferred cutting tool has been described for forming an undersized opening, it is recognized that a variety of structurally different hole forming devices can be used. That is, although the description thus far has discussed the device key dimension in terms of its width or outer diameter, it is recognized that alternatively, the device key dimension could be discussed in terms of its circumference, or more generally, in terms of its periphery. Regardless of the terminology used, it is important in accordance with the invention, that the opening formed by the physician be undersized relative to the device periphery so that the surrounding tissue remains stressed after device insertion. Accordingly, it should be understood that the cutting member need only include a cutting edge configured to form an opening (which is typically circular but can be of any other shape) whose periphery is dimensioned to require stretching of the surrounding tissue to accommodate the device periphery.

1. A method of implanting a medical device into the soft tissue of a patient where the device carries a porous layer having a dimension W, comprising the steps of:
forming an opening into said soft tissue which opening has a dimension less than W when the tissue surrounding said opening is relaxed;
stretching said surrounding tissue to enlarge said opening to allow said device to be inserted therethrough; and
terminating said stretching to allow said surrounding tissue to relax against said porous layer in a stressed state to promote tissue ingrowth into said porous layer.

2. The method of claim 1 wherein said opening defines a maximum dimension of 0.9W.

3. A cutting tool configured to cut an opening into a patient's soft tissue for receiving a medical device carrying a porous layer which has a width dimension W, said cutting tool comprising:
a handle;
a blade projecting from said handle, said blade defining a cutting edge having a width dimension less than W.

4. The cutting tool of claim 3 wherein said cutting edge includes a pointed front end and first and second edge portions diverging rearwardly from said front end.

5. The cutting tool of claim 4 wherein said first and second edge portions diverge rearwardly to define a maximum width of 0.9W.

6. The cutting tool of claim 3 wherein said handle defines a stop for limiting the depth of penetration of said cutting edge into said patient's soft tissue.

7. A cutting tool configured to cut an opening into a patient's soft tissue for receiving a medical device carrying a porous layer defining a device periphery, said cutting tool comprising:
a cutting member defining a cutting edge for forming an opening having a periphery dimension less than said device periphery.

8. The cutting tool of claim 7 wherein cutting edge maximum periphery dimension is 90% of said device periphery.
